(19) United States
(12) Patent Application Publication
(10) Pub. No.: US 2025/0331772 A1
(43) Pub. Date: Oct. 30, 2025

(54) OPIOID DETECTION

(71) Applicant: PercuSense, Inc., Valencia, CA (US)

(72) Inventors: Rajiv Shah, Rancho Palos Verdes, CA (US); Bradley Liang, Bloomfield Hills, MI (US); Brian Kannard, Los Angeles, CA (US); Katherine Wolfe, Dunwoody, GA (US); Blake Morrow, Los Angeles, CA (US); Kaitlynn Olczak, Valencia, CA (US)

(73) Assignee: PercuSense, Inc., Valencia, CA (US)

(21) Appl. No.: 19/040,823

(22) Filed: Jan. 29, 2025

Related U.S. Application Data
(60) Provisional application No. 63/626,572, filed on Jan. 30, 2024.

(51) Int. Cl. A61B 5/00; A61B 5/0537; A61B 5/145; A61B 5/1473; A61B 5/1486

(52) U.S. Cl. CPC A61B 5/4845; A61B 5/0004; A61B 5/0537; A61B 5/1451; A61B 5/14532; A61B 5/14546; A61B 5/1473; A61B 5/14865; A61B 5/4875; A61B 5/7435; A61B 5/746

(57) ABSTRACT

A multi-analyte sensor system is disclosed that includes a sensor probe with a first set of electrodes that transduce choline into electrical signals and a second set of electrodes that transduce tissue oxygen into electrical signals. The sensor probe includes a third set of electrodes that provide working and counter electrode functionality for the first and second set of electrodes. The system also has an electronics module interfaced with the sensor probe that includes a transceiver to transmit sensor data. The system further includes control circuitry that determines a choline state based on the first set of electrodes and determines a tissue oxygen state based on the second set of electrodes. The control circuitry is also configured to initiate an alarm condition based on one or more signals from the first and second set of electrodes.

### RELATED APPLICATIONS

This application claims the benefit of U.S. provisional application No. 63/626,572 filed on Jan. 30, 2024, 63/707,724, filed Oct. 15, 2024, and 63/725,486, filed on Nov. 26, 2024. The applications listed above are hereby incorporated by reference in their entireties for all purposes.

### BACKGROUND

The present disclosure generally relates to systems, devices, and methods for real time monitoring of physiological parameters to enable monitoring of physical conditions or physical states. More specifically, the present disclosure relates to the use of sensors and related control circuitry to enable detection of opioid exposure and efficacy of countermeasures of opioid exposure.

It may be highly desirable to develop systems that are capable of detecting both exposure to opioids and the efficacy of countermeasures to opioid exposure. While embodiments and examples discussed in detail below may be related to particular analytes and physical parameters, the scope of the disclosure and claims should not be construed to be limited to the specifically addressed analytes and parameters associated with metabolic health and diabetes. Rather, it should be recognized that additional/other analytes and/or physical parameters can be monitored to assist in the detection and diagnosis of various conditions or general physiological health.

### BRIEF SUMMARY OF THE INVENTION

In one embodiment a multi-analyte sensor system is disclosed that includes a sensor probe that has a first set of electrodes with one or more first working electrodes configured to transduce choline into electrical signals. The sensor probe also includes a second set of electrodes with one or more second working electrodes configured to transduce tissue oxygen into electrical signals. The sensor probe also has a third set of electrodes with one or more third electrodes that provide working and counter electrode functionality for the first set of electrodes and the second set of electrodes. The system also has an electronics module that electrically interfaces with the sensor probe, the electronics module including a transceiver configured to transmit sensor data. The system further includes control circuitry communicatively coupled to the electronics module. The control circuitry configured to determine a choline state based on one or more signals from the first set of electrodes and determine a tissue oxygen state based on one or more signals from the second set of electrodes. The control circuitry also configured to initiate an alarm condition based on the one or more signals from the first set of electrodes and the one or more signals from the second set of electrodes.

In another embodiment, a continuous multianalyte monitoring system is disclosed that includes a skin-mounted sensor control unit that has a percutaneous multianalyte sensor that has an insertion portion configured for transcutaneous positioning in a subcutaneous tissue of a user. The percutaneous multianalyte sensor is configured to sense levels of choline and tissue oxygen in the subcutaneous tissue of the user. The system also includes an adhesive patch disposed on a bottom surface of the skin-mounted sensor control unit and configured to adhere the skin-mounted sensor control unit to skin of the user. The system further has a transceiver configured for wireless communication with the skin-mounted sensor control unit. Additionally, the system includes control circuitry configured to receive and store signals from the percutaneous multianalyte sensor related to sensed levels of choline and tissue oxygen and determine a real-time physical condition value based on the received signals related to sensed levels of choline and oxygen. The control circuitry is also configured to determine whether the user has been exposed to an opioid based on the real-time physical condition value and generate user interface data for rendering on a touch-interface display to visually display a graph of the choline and tissue oxygen levels, the graph representing a first axis corresponding to time, a second axis corresponding to one or more of the choline levels or tissue oxygen levels, and the physical condition value.

In another embodiment, a multi-analyte sensor system is disclosed that includes a sensor probe that has a first set of working electrodes configured to transduce choline into first electrical signals and a second set of working electrodes configured to transduce tissue oxygen into second electrical signals. The sensor probe further includes a third set of electrodes that are configured to provide reference and counter electrode functionality for the first set of working electrodes and the second set of working electrodes. The system includes an electronics module configured to electrically interface with the sensor probe where the electronics module includes a transceiver configured to wirelessly transmit sensor data. The system further includes control circuitry communicatively coupled to the electronics module and configured to store threshold values that are indicative of opioid exposure. The control circuitry also determines a choline state based on one or more signals from the first set of working electrodes and determine a tissue oxygen state based on one or more signals from the second set of working electrodes. The control circuitry further determines an opioid condition based on at least the choline state and the tissue oxygen state, and initiate an alarm condition based on the opioid condition.

Other features and advantages of the invention will become apparent from the following detailed description, taken in conjunction with the accompanying drawings that illustrate, by way of example, various features of embodiments of the invention.

Methods and structures disclosed herein for treating a user/patient also cover analogous methods and structures performed on, or placed on, a simulated patient, which can be useful, for example, for training, demonstration, procedure and/or device development, and the like. For example, a simulated patient can be physical, virtual, or a combination of physical and virtual. A simulation can include a simulation of all or a portion of a patient, such as an entire body, a portion of a body, a system, an organ, or any combination thereof. Physical elements can be natural, including human or animal cadavers, or portions thereof; synthetic; or any combination of natural and synthetic. Virtual elements can be entirely in silicon, or overlaid on one or more of the physical components. Virtual elements can be presented on any combination of screens, headsets, holographically, projected, loudspeakers, headphones, pressure transducers, temperature transducers, or using any combination of suitable technologies.

### BRIEF DESCRIPTION OF THE DRAWINGS

FIG. 1 is an exemplary block diagram showing components of a system configured to detect and process signals and/or data sets indicative of at least one physiological state of a subject 1 (e.g., exposure to opioids) in accordance with embodiments of the invention.

FIG. 2 is an exemplary block diagram of a device that integrates into a single device the previously discussed sensor system.

FIGS. 3A-3D are exemplary block diagrams illustrating various electrode configurations of analyte sensor probes 304a-304d, in accordance with various embodiments of the present disclosure.

FIGS. 4A and 4B are exemplary views of an A-side and a B-side of an implantable probe, also referred to as an analyte sensor or sensor probe, that includes the sensor array, in accordance with embodiments of the invention.

FIG. 4C is an exemplary cross-section illustration of the analyte sensor illustrating diffusion of analyte, reactant and reaction by-product within one working electrode in accordance with embodiments of the present invention.

FIGS. 5A and 5B are an exemplary illustration of A-side and B-side of a sensor probe, in accordance with other embodiments of the present invention.

FIGS. 6A and 6B are an exemplary illustration of A-side and B-side of a sensor, in accordance with another embodiment of the present invention.

FIG. 7 is an exemplary illustration of real-time choline, lactate, and tissue oxygen data illustrative of intentional exposure to an opioid and subsequent delivery of an opioid countermeasure, in accordance with embodiments of the present invention.

FIG. 8 is an exemplary flowchart illustrating one embodiment of detecting a physiological condition, in accordance with various embodiments of the present invention.

### DETAILED DESCRIPTION

Automated opioid detection can improve response time administration of countermeasures and additionally provide quantitative metrics that are indicative of efficacy of opioid countermeasures. Example systems of the present disclosure utilize real-time multianalyte sensing to detect exposure to, and efficacy of treatment for, exposure to opioids.

Controlled use of opioids can be used effectively to control pain but accidental exposure can have potentially deadly consequences. Potential victims of accidental opioid exposure include first responders such as law enforcement or fire fighters. Additionally, military personnel may encounter opioids during drug interdiction operations. Accordingly, there is a need to be able to quantitatively determine if a subject has been exposed to opioids. Moreover, in addition to detection of opioid exposure, there is a need to determine if countermeasures to opioid exposure are having the desired effect.

In preferred embodiments real-time monitoring of one or more analytes within the subject enables detection of opioid exposure. In some embodiments choline is an analyte that is monitored to determine opioid exposure. In other embodiments, in addition to choline, a second analyte may also be monitored. It may be beneficial to monitor the second analyte in order to reduce the likelihood of false positives of opioid exposure from the single analyte. In many of these embodiments the second analyte is tissue oxygen. In these embodiments, the first and second analytes may be measured or detected within the interstitial fluid, or microcirculation, of the subject. Accordingly, in many embodiments the analyte or analytes are detected or measured using a sensor that is implanted percutaneously into the subcutaneous tissue of the subject.

In some embodiments, the detection of physiological conditions associated with opioid exposure is accomplished using a combination of biochemical signals associated with choline and oxygen. The biochemical signals can be derived from a minimally-invasive probe used to produce a continuous signal representative of one or both of choline and tissue oxygen. The ability to measure multiple biochemical signals via a single probe results in a system that reduces burden on the subject rather than requiring mindfulness of multiple sensor insertions. The seamless integration of multiple signal streams can enable multiple days of opioid detection. Such integration can further enable rapid individualization optimization efforts from the additional time series data generated and the data that can be distilled from the interaction between signal streams.

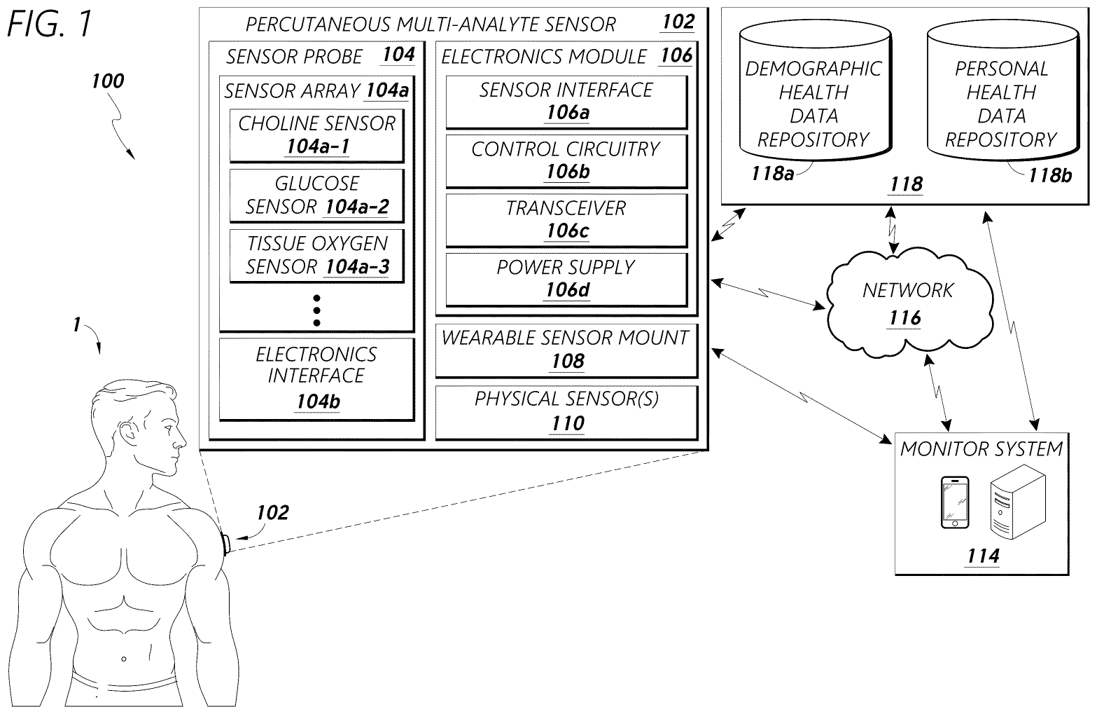

FIG. 1 is an exemplary block diagram showing components of a system 100 configured to detect and process signals and/or data sets indicative of at least one physiological state of a subject 1 (e.g., exposure to opioids) in accordance with embodiments of the invention. The system 100 advantageously provides a technical improvement for opioid detection using an analyte sensor by integrating multiple analyte-sensing and data-processing functions. Broadly, the system 100 includes a percutaneous multi-analyte sensor system 102 that includes a sensor probe 104 that is electrically coupled to an electronics module 106 via an electronics interface 104b. The sensor probe 104 advantageously captures multiple analyte signals (e.g., choline and tissue oxygen) at a single insertion site, thereby reducing patient discomfort compared to multiple separate sensors. Optionally, a sensor mount 108 and one or more physical sensors 110 (e.g., accelerometers, thermometers) may be included within the sensor system 102. Collectively, these components provide a hardware-based platform capable of continuous, real-time measurements for improved metabolic state determinations.

In preferred embodiments, the analyte sensor 104 is an electrochemical sensor probe that includes a sensor array 104a configured to measure/detect specific molecules of interest in vivo. Using specialized electrode configurations, the sensor array 104a can implement electrochemical sensing to simultaneously measure concentrations of choline, tissue oxygen, and/or one or more additional analytes. For example, a choline sensor 104a-1 of the sensor array 104 can be configured to implement amperometric detection with a selective enzyme coating of choline oxidase, whereas a tissue oxygen sensor 104a-3 can be configured to amperometrically detect oxygen within interstitial fluid. This approach advantageously leverages real-time biochemical measurements to determine dynamic physiological states, such as exposure to opioids and any physiological response to opioid countermeasures.

In some embodiments, the sensor array 104a further includes the capability or option to detect or measure an optional third analyte of molecule of interest. For example, as illustrated in FIG. 1, the sensor array 104a includes an optional glucose sensor 104a-2. The illustration in FIG. 1 of the choline sensor 104a-1, the oxygen sensor 104a-3, and the glucose sensor 104a-2 should not be construed as limiting. In some embodiments, the sensor array 104a can include additional sensors to detect or measure other molecules or analytes of interest such as, but not limited to ketone sensors using potentiometric methods, reactive oxygen species (ROS) sensors using chronoamperometry, and/or sensors to detect/measure lactate, acetylcholine, alcohol and/or the like. Any such additional analyte sensors can be integrated to further enable detection of metabolic conditions such as ketosis or oxidative stress.

The electronics interface 104b facilitates electrical communication between the analyte sensor 104 and the electronics module 106. While illustrated as part of the sensor probe 104, in other embodiments the electronics interface 104b may be embodied at least in part in the electronics module 106. As the electronics interface 104b is intended to interface between the analyte sensor 104 and the electronics module 106, its relative association or location between the elements or components within the sensor system 102 should not be construed as limiting.

In some embodiments, the electronics module 106 includes a sensor interface 106a, a communication module (e.g., transceiver) 106c, and/or a power supply 106d. In some implementations, the sensor interface 106a is configured to enable electrical coupling between the electronics module 106 and the electronics interface 104b. The sensor interface 106a can be configured to enable electrical signals generated by the analyte sensor 104 to be transmitted to the control circuitry 106b.

The electronics module 106 further includes additional control circuitry 106b in addition to the sensor interface 106a, communications/transceiver circuitry 106c, and power supply circuitry 106d, wherein the control circuitry 106b may be configured to perform certain signal processing, amplification, filtering, conversion, calibration, and management/control functions for the sensor 102. In preferred embodiments the control circuitry 106b may include, but is not limited to elements such as clocks, memory, processors, analog-to-digital converters and the like. Such components can enable real-time signal processing, including filtering, amplification, and transformation of raw electrochemical signals into calibrated glucose and lactate concentrations. For example, the processor applies adaptive algorithms to correct for temperature variations or cross-analyte interference, ensuring accurate real-time data for physiological state determination. By integrating these functions within a single hardware platform, the system 100 offers enhanced reliability and responsiveness, supporting improved safety and efficacy in automatic detection of opioid exposure.

The control circuitry 106b may be configured to enable control of the analyte sensor 104. The control circuitry 106b can further enable data processing of signals generated or detected by the analyte sensor 104. For example, in many embodiments, the control circuitry 106b can be configured to apply machine-learning models trained on personal, demographic, and/or historical data to dynamically adjust thresholds related to the detection of opioids. For example, in some embodiments, the control circuitry 106b enables transformation of raw signals from the analyte sensor 104 to be representative of the respective molecule or analyte being detected. The control circuitry 106b can further generate and/or display information that is more meaningful for the subject than analyte or molecular concentrations. The terms “circuitry” and “control circuitry” are used herein according to their broad and ordinary meanings, and may refer to any individual or collection of processors, processing circuitry, processing modules/units, chips, dies (e.g., semiconductor dies including come or more active and/or passive devices and/or connectivity circuitry), microprocessors, micro-controllers, digital signal processors, microcomputers, central processing units, field programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Circuitry referenced herein may further comprise one or more storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in examples in which circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

Further included in the electronics module 106 is a communication module (e.g., transceiver) 106c. The communication module 106c enables communication between the sensor system 102 and other components within the system 100. In many embodiments the communication module 106 enables two-way communication via any suitable or desirable communication protocol(s), such as, but not limited to Wi-Fi, Bluetooth or wireless network technologies such as 4G, 5G and the like. The communication module 106c enables data acquired by the sensor system 102 to be transmitted in either raw, partially processed, or fully processed to other components within the system 100. Additionally, the communication module 106c further enables data from other components within the system 100 to be used as input for the sensor system 102. For example, in many embodiments the communication module 106c enables data from an electronic health record to be dynamically input into the control circuitry of the sensor system 102. A power supply 106d is also included as part of the electronics module 106. In preferred embodiments the power supply 106d is an energy storage device such as a disposable or rechargeable battery. In alternative embodiments the power supply 106d may include or be based on energy storage technologies such as, but not limited to solar cells, capacitors, fuel cells and the like.

In some embodiments, the sensor system 102 optionally includes a sensor mount 108, which may be attached or coupled to the subject/user 1 and is configured to receive and secure the electronics module 106. In many embodiments, the sensor probe 104 may be coupled or attached to the sensor mount 108. Insertion of the sensor probe 104 may serve to couple the sensor mount 108 to the subject. After insertion of the sensor probe 104, the electronics module 106 may be coupled to the sensor mount 108 and begin providing power to the sensor probe 104. In some embodiments, the electronics module 106 may be removably coupled to the sensor mount 108, such that the electronics module 106 may be reusable in its entirety, which may be particularly advantageous for embodiments that utilize a rechargeable or replaceable power supply. In other embodiments, portions of the electronics module 106 may be reused or recycled to reduce overall electronic waste.

In many embodiments, one or more physical sensors 110 may be optionally included as part of the sensor system 102. The inclusion of physical sensor(s) 110 can enable detection of parameters that can affect the integrity or validity of data acquired via the sensor probe 104. Exemplary, non-limiting physical sensors that may be integrated within the sensor system 102 include, but are not limited to, accelerometers, thermometers and the like, which can provide additional insights regarding physiological conditions of the subject. The inclusion of optional physical sensors 110 can provide insight associated with metabolic health and/or aerobic health. For instance, accelerometer data can indicate periods of sustained motion corresponding to exercise or movement.

The system 100 further includes a data repository 118. The data repository 118 can advantageously store data that can influence or have an impact on data provided by the sensor system 102. Exemplary data retained in the data repository 118 can include, but is not limited to, data indicating attributes of the subject 1 and/or demographic population(s) relating age, gender, height, weight, body mass index, waist circumference, blood pressure (diastolic or systolic), cholesterol, any and quantities of both chronic and acute medications, along with any chronic conditions. The exemplary data described above that can be stored in the data repository 118 should not be construed as limiting. In preferred embodiments, any type of health metric that may be recorded in an electronic or physical health record may be input and stored in the data repository 118. The demographic and/or personal health data can enable the system to contextualize real-time sensor outputs.

In many embodiments, the data repository 118 includes a demographic data repository 118a and a personal data repository 118b. In some implementations, the demographic health data 118a and the personal health data 118b are stored in the same physical data storage device(s) or server(s). Both the demographic data repository 118a and the personal data repository 118b may store data of a similar type. However, in preferred embodiments, the demographic data repository 118a is anonymized, while the personal data repository 118b is specific to a particular user or subject. The demographic data repository 118b can enable analysis of data across various demographics represented by the data stored therein. For example, in some embodiments, the demographic data repository 118a enables artificial intelligence or other trainable model configured to analyze the data for patterns or trends that can be applied to modify or control other components within the system 100.

A network 116 is included within the system 100 to enable communication between various components within the system 100. The network 116 may leverage various communication protocol(s), such as cellular or mobile networks (e.g., 5G, 4G and the like), Wi-Fi, Bluetooth, Zigbee and/or the like. The network 116 can enable data from the sensor system 102 to be stored in the data repository 118. Additionally, the network 116 can enable the use of data stored in the demographic data repository 118a and/or personal health data repository 118b as input to control or modify control of other components within the system 100.

The system 100 further includes a monitoring system 114, which may be local, remote, or both. In preferred embodiments, the monitor system 114 leverages the network 116 to communicate with other components within the system 100. In some embodiments, the monitor system 114 includes the ability to process data from the various components within the system 100. For example, in some embodiments the monitor system 114 can receive data from the sensor system 102 as raw data and process the raw data to be representative of the respective analytes or molecules. In some embodiments, the monitor system 114 is configured to receive processed data from other components within the system 100. In some such embodiments, the monitor system 114 can supplement the processed data with data from other components and further transform the data.

Transformation of data from the sensor system 102 enables determination of secondary considerations or conditions based on, or derived from, real-time measurements from the sensor system 102. “Secondary conditions,” as described herein, may be any physiological or metabolic states that are derived from or influenced by primary data (e.g., glucose levels, lactate levels, and/or oxygen levels) measured by a sensor (e.g., multi-analyte sensor) of a system. Secondary conditions can comprise higher-order conditions inferred from the integration of primary analyte data with other inputs (e.g., activity, medication, chronic health status), and can provide insights into the subject's metabolic health and/or guide adjustments to therapeutic interventions. Secondary conditions can include data structures representing any of exemplary conditions such as insulin resistance, metabolic stress, fasting state, postprandial state, chronic disease impact, hypoxic or oxygen-deprived states, medication interactions, or the like.

In some embodiments, the monitor system 114 includes a display. In preferred embodiments, the monitor system 114 displays data from various components of the system 100, such as analyte levels detected by the sensor system 102. Inputs from the data repository 118 can be processed by an artificial intelligence module within the system 100, which is configured to apply machine learning algorithms to analyze historical and real-time data from the sensor system 102. In still other embodiments, based on predictive data, the system 100 can determine and display on the monitor system 114 recommendations to the user to improve predictive data relative to long or short term goals or objectives associated with the metrics from components within the system 100. Exemplary, non-limiting embodiments of the monitor system 114 include, but are not limited to systems such as mobile phones, tablets, laptop computers, desktop computers, vehicle infotainment systems, home automation systems, and the like.

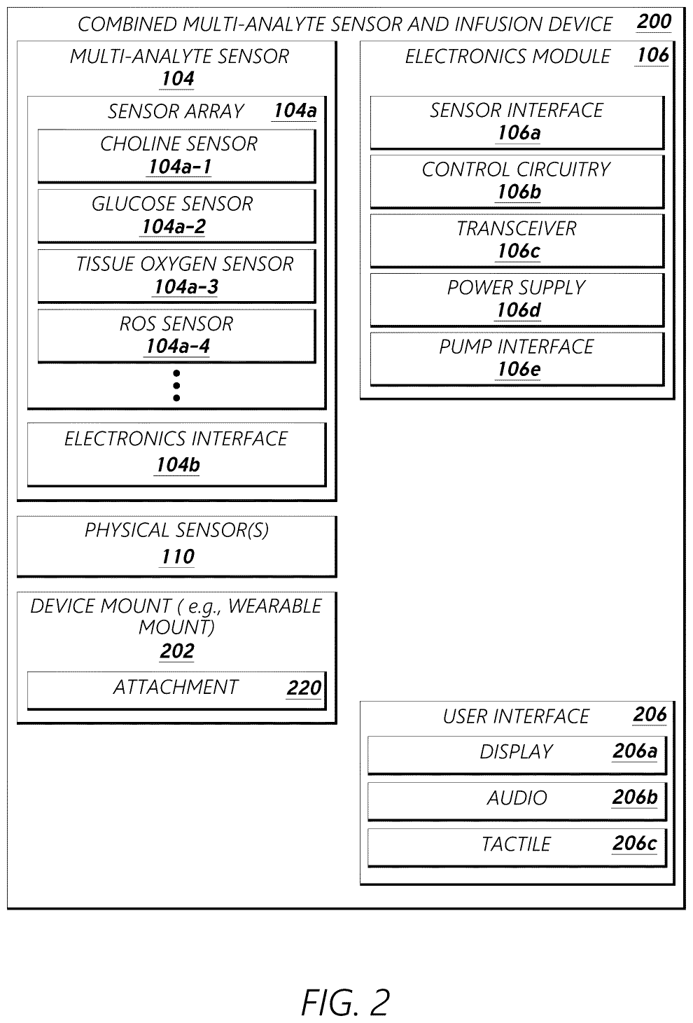

FIG. 2 is an exemplary block diagram of a device 200 that integrates into a single device the previously discussed sensor system 102. With respect to inventive multi-analyte systems and processes disclosed herein, the sensor system 102 can provide technical improvements with respect to the technical challenge of simultaneously monitoring real-time analyte levels and further provides technical improvements with respect to reduced insertion sites, simplified user operation, and improved data fidelity from co-located sensors.

The device 200 includes the analyte sensor 104 having the sensor array 104a. Similar to FIG. 1, the sensor array 104a may include the choline sensor 104a-1, the glucose sensor 104a-2, and/or the tissue oxygen sensor 104a-3. The specific analytes described above and illustrated within the sensor array 104a should not be construed as limiting. The sensor array 104a of the analyte sensor 104 may be configured to detect or measure more or fewer analytes of interest. Moreover, the sensor array 104 may be configured to detect or measure additional or different analytes than those described herein. For example, in some embodiments, any molecule capable of being detected or measured electrochemically may be implemented within the sensor array 104a.

The sensor array 104a includes the electronics interface 104b that enables the analyte sensor 104 to be coupled with the electronics module 106. The device 200 can further include physical sensors 110, along with a device mount 108. The device mount 108 enables the device 200 to be removably mounted or applied to a subject. The device 200 additionally includes the electronics module 106 that has the sensor interface 106a, the control circuitry 106b, the communication module 106c, and the power supply 106d.

The device 200 may be removably coupled or secured to a subject using a device mount 202. In some embodiments, the device mount 202 includes a base to which the analyte sensor probe 104 is attached and placement of the device mount 202 on the subject coincides with insertion of the analyte sensor probe 104 within the subject. In such embodiments, addition of the remaining components to the device mount 202 can complete the device 200.

The device 200 further includes a user interface 206. The user interface 206 may include one or more visual display components 206a, audible components 206b, and/or tactile components 206c. In some embodiments, the visual/display component(s) 206a can include one or more lights that indicate a status of the device 200. Similarly, the audio component(s) 206b may include one or more piezoelectric devices or other sound-emitting device configured to produce audible sounds or sequences of sounds to convey or indicate a status of the device 200. Likewise, the tactile component(s) 206 may include vibration devices that create a tactile sensation or sequence of tactile sensations to convey or indicate a status of the device 200.

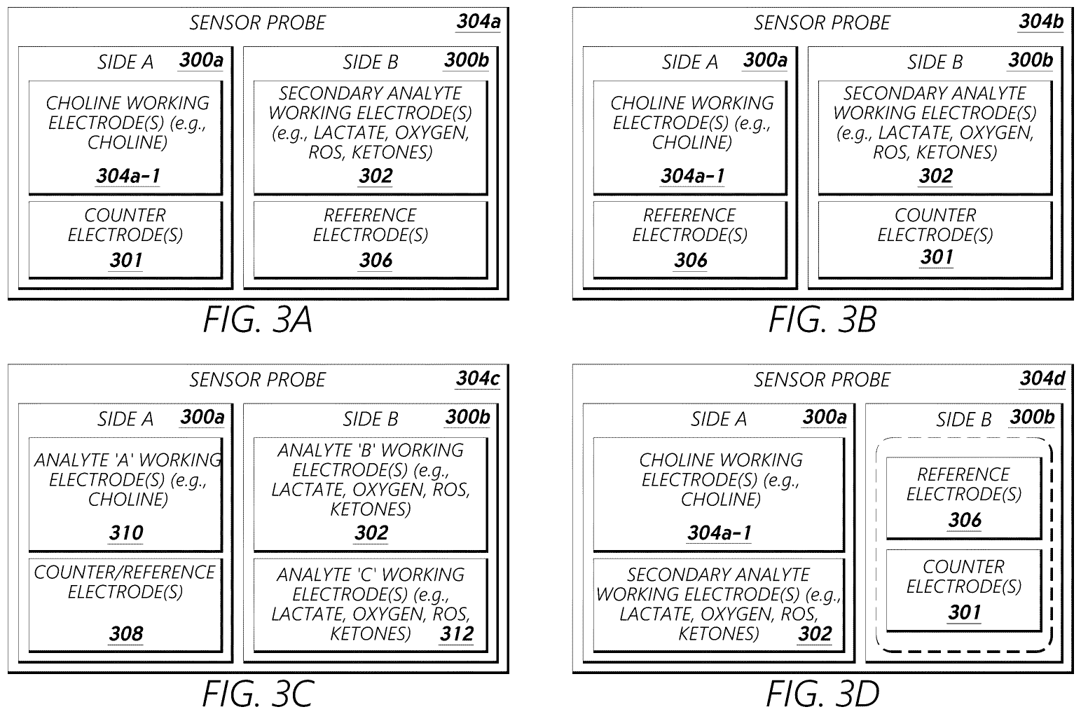

FIGS. 3A-3D are exemplary block diagrams illustrating various electrode configurations of analyte sensor probes 304a-304d, in accordance with various embodiments of the present disclosure. Example analyte sensor probes of the present disclosure may have configurations of any of the analyte sensor probes 304a-304d. The analyte sensor probes 304a-304d illustrated in FIGS. 3A-3D each include two sides/faces, namely an ‘A-side’ 300a and a ‘B-side’ 300b. For example, the sensor probes 304a-304d can have a thin, flat, elongated body with a generally rectangular cross-section, wherein the probes 304a-304d have two relatively broad, flat faces on opposite sides, which are parallel and define the primary surface area of the probe. Edges may generally run along the length of the probes 304a-304d where the two faces meet, forming two elongated, relatively narrow surfaces. Sides ‘A’ and ‘B’ of the sensor probes 304a-304d may correspond to the opposite-facing primary surfaces/faces of the respective probes. By implementing two-electrode or three-electrode systems (or hybrid configurations) on a single, thin, elongated sensor probe, these embodiments enable multi-analyte sensing while minimizing patient discomfort and maximizing measurement accuracy.

Each side of the analyte sensor probes 304a-304d may be configured with one or more electrodes, wherein each electrode can be configured as one of a working electrode, a counter electrode, a reference electrode, or a combined counter and reference electrode. In some embodiments, a two-electrode configuration is implemented, wherein a working electrode is operated with a combined counter and reference electrode. In other embodiments, a three-electrode configuration may be implemented, wherein a working electrode is operated with a separate counter electrode and a separate reference electrode. In some embodiments of a two-electrode system, multiple working electrodes operated with the same polarity may share a combined counter and reference electrode. Similarly, in some embodiments of a three-electrode system, multiple working electrodes operated with the same polarity may share a counter electrode and a reference electrode.

FIG. 3A is an exemplary illustration of a three-electrode sensor probe configuration 304a, wherein the A-side 300a includes a choline working electrode 304a-1 and one or more counter electrodes 301. The B-side 300b is configured with a second working electrode 302 configured to measure or detect a second analyte (e.g., oxygen or lactate) and further includes one or more reference electrodes 306.

FIG. 3B is an exemplary illustration of a three-electrode sensor probe configuration 304b, wherein the A-side 300a includes a choline working electrode 304a-1 and one or more reference electrodes 306. The B-side 300b is configured to measure or detect a second analyte 302 and further includes one or more counter electrodes 304.

FIG. 3C is an exemplary illustration of a two-electrode sensor probe configuration 304c, wherein the A-side 300a is configured with a first working electrode to measure a first analyte 310 and further includes a combined counter and reference electrode. The B-side 300b is configured with a second working electrode 302 and a third working electrode 312. In various embodiments, the second working electrode 302 may be configured to detect the same or a different analyte than the first working electrode. Similarly, the third working electrode 312 may be configured to detect the same or a different analyte than the first working electrode 310 and/or the second working electrode 302.

FIG. 3D is an exemplary illustration of a three-electrode sensor probe configuration 304d, wherein the A-side 300a includes a choline working electrode 304a-1 and a second working electrode 302. The B-side 300b is configured with one or more reference electrodes 306 and one or more counter electrodes 304. The permutations illustrated in FIGS. 3A-3D and described above should be construed as exemplary rather than limiting. For example, in some embodiments a two-electrode system may be implemented on the same analyte sensor 104 as a three-electrode system. In such an embodiment the first and second working electrodes may be formed on the A-side while the B-side includes a combined counter and reference electrode along with a discrete counter electrode and a discrete reference electrode.

Any of the example configurations of FIGS. 3A-3D may be implemented in connection with embodiments of the present disclosure. In a given implementation, the positions of the electrodes may advantageously be optimized for accurate multi-analyte sensing by leveraging the geometry of the probe and the electrochemical requirements of each electrode. For example, the choline electrode may desirably be positioned near the tip on the A-side, ensuring direct exposure to interstitial fluid in highly vascularized regions for consistent choline measurements. The oxygen electrode may also be positioned on the A-side, located slightly proximal to the choline electrode, allowing it to measure oxygen levels downstream from choline uptake. In alternative embodiments, an oxygen electrode may be positioned near the middle of the B-side of the probe, separated from working electrodes for other analytes to avoid interference from their electrochemical reactions while providing critical oxygen measurements that contextualize metabolic activity. The reference electrode may be placed centrally along the probe's B-side, such as equidistant from the working electrodes on both sides, to maintain a stable potential across all measurements. The counter electrode, where separate from the reference electrode, may be positioned at the proximal end of the B-side, providing uniform current flow for all working electrodes while minimizing interference with analyte detection. This example beneficial arrangement, and/or aspects or considerations associated therewith, can advantageously provide for reduced cross-interference, stable signal acquisition, and/or efficient use of the probe's surface area, enabling robust, synchronized multi-analyte sensing with a single implantable device.

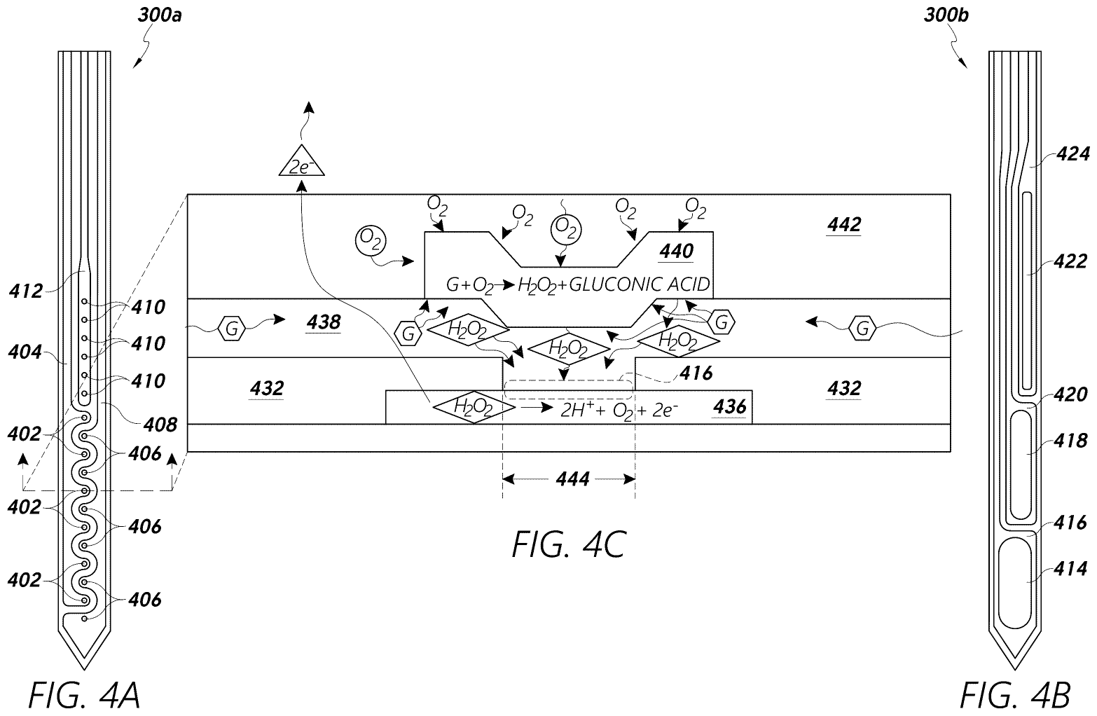

FIGS. 4A and 4B are exemplary views of an A-side 300a and a B-side 300b of an implantable probe, also referred to as an analyte sensor or sensor probe, that includes the sensor array 104a, in accordance with embodiments of the invention. In varying embodiments, the sensor array 104a includes an A-side 300a and a B-side 300b that enables continuous detection of choline and at least a second analyte, such as oxygen. While some embodiments of the analyte sensor 104a use both A-side 300a and B-side 300b, other embodiments utilize only a single side. In addition to choline, exemplary additional analytes that can be measured on the A-side 300a, B-side 300b, or across both A-side 300a and B-side 300b, include, but are not limited to, lactate, glucose, reactive oxygen species (ROS), ketones, and the like. While illustrated as a single probe, varying embodiments of the sensor array 104a include multiple probes, each capable of measuring identical or different analytes using different combinations of working electrodes. Examples of a sensor array 104a having multiple probes but a single point of entry can be found in combined sensing and infusion devices discussed in U.S. patent application Ser. No. 15/455,115 filed on Mar. 9, 2017 which is hereby incorporated for reference for all purposes.

In some embodiments, the analyte sensor 104, or implantable probe, can be implanted via a surgical procedure. In other embodiments, the analyte sensor 104 can be temporarily inserted into tissue, such as, but not limited to subcutaneous tissue, muscle tissue, organ tissue, or the like. In some embodiments, the implantable probe 104 may be temporarily inserted into tissue for varying durations that can be measured in minutes, hours, days, weeks, or months. While many embodiments of the implantable probe, or analyte sensor, 104 have been discussed as using both an A-side 300a and B-side 300b, other embodiments utilize a single side of the implantable probe.

FIG. 4A is a view of the A-side 300a that includes first working electrodes 402 and second working electrodes 406 along with corresponding first electrode trace 404 and second electrode trace 408. In many embodiments the first working electrodes 402 are transducers configured to detect, or measure, choline concentration. The second working electrodes 406 can be configured to measure the concentration of a second analyte such as, but not limited to oxygen, lactate, ROS, ketones, or the like. FIG. 4B is a view of the B-side 300b that includes a plurality of combination counter-reference electrodes 414 and 418 formed on electrode traces 416 and 420 respectively. In preferred embodiments a two-electrode system consisting of the first and second working electrodes with corresponding combined counter-reference electrodes, or pseudo-reference electrodes, are used to detect concentrations of the various analytes. However, other embodiments may use a three-electrode system having a working electrode along with a counter electrode and a reference electrode.

FIGS. 4A and 4B further include optional third working electrodes 410 formed on third electrode trace 412 in addition to third counter-reference electrode 422 formed on electrode trace 424. In some embodiments, the third working electrodes 410 can be duplicative of the second working electrodes 406, such that the third working electrodes 410 measure or detect the same analyte as the second working electrodes 406. However, in other embodiments, the third working electrodes 410 are used to measure a third analyte, such that the analyte sensor 104 (see FIG. 1) is capable of measuring choline, and at least two of oxygen, lactate, ROS, ketones or the like. Common among the embodiments is at least measuring choline because choline measurements can assist in detecting opioid exposure. The addition of the second analyte such as oxygen and optional third analyte are intended to supplement choline and provide additional certainty regarding opioid exposure.

Measurement of some physical characteristics can be enabled via physical sensors or other instrumentation incorporated within or on the electronics module. With further reference to FIG. 1, in many embodiments, detecting a physiological state is accomplished via a combination of the analyte sensors 104 implanted within the subject and the physical sensors 110 associated with the electronics module 106. The specific physical sensors 110 discussed should not be construed as limiting. Other and additional physical characteristics from physical sensors 110 associated with the sensor system 102 can be used as inputs to detect or confirm various physiological states. Monitoring hydration levels of at least one, some, or all of the transducers within the sensor array 104a enables detection of whether the sensor array 104a is properly implanted within desirable tissue. Additionally, monitoring the sensor elements for proper hydration can be used as a trigger to enable at least one of determining or detecting a physiological state, data recording, and/or data transmission.

Electrochemical impedance spectroscopy (EIS) applied across any electrode pair within the sensor array 104a can be used to measure or infer tissue impedance to determine tissue hydrations levels, or a fluid status within subcutaneous tissue of a subject being monitored. Utilizing the sensor array enables continuous monitoring of tissue impedance to detect changes in fluid content within the interstitial space. In some embodiments, an EIS scan across specific frequencies is used to correlate impedance with sodium concentrations within tissue surrounding the sensor via either a lookup table or via an equivalent circuit model. Regardless of how impedance is determined, real-time monitoring can enable EIS measurements over time to determine if there is an increase in fluid within interstitial fluid based on changes in salinity of the interstitial fluid. If salinity decreases, one can infer there is additional fluid buildup. Conversely, if salinity increases, it can be inferred that the fluid level within the interstitial fluid is decreasing. Increased, or increasing fluid within interstitial fluid results in lower relative impedance, measurable across multiple frequencies.

Rapid changes in tissue impedance may be correlated with changes in hydration which can be correlated to detecting a physiological state such as, but not limited to sleep, exercise, strenuous exercise and meal intake. In some embodiments, fluid status or hydration of a subject contributes to detecting a physiological state because fluid status provides context and a normalizing factor for other measurements, such as, but not limited to tissue oxygen levels and concentrations of ROS. Absolute and trend information derived from tissue hydration levels enable adjustment or modifications to detecting a physiological state. In some embodiments, tissue hydration levels enable additional insight regarding perfusion of analytes within different types of tissues. For example, in various embodiments tissue hydration levels for a sensor array 104a placed in muscle provides additional or less information than a sensor array 104a that is placed in adipose tissue.

With continued reference to FIG. 1, detecting a physiological state can be accomplished by analyzing real-time data from the sensor system 102 for trends in the data that are indicative of a physiological state. In some embodiments, detecting a physiological state is based on data from the sensor array 104a exceeding a threshold value. In some embodiments, the threshold values for various physiological states are associated with data from only the analyte sensors 104. In other embodiments, the threshold values for some physiological states are associated with data from the analyte sensors 104 and/or the physical sensors 110. In still other embodiments, threshold values for physiological states are set based on data from the physical sensors 110. Threshold values can be associated with absolute changes or rates of change of a single analyte, multiple analytes with or without additional absolute changes or rates of change data from the physical sensors 110. In still other embodiments, detecting a physiological state is based on a change in data from the sensor array relative to historical data from the sensor array. Typical changes in data that may be detected include, but are not limited to, changes in value, rate, coefficient of variance, and the like.

Once a threshold value for a physiological state has been crossed, a probability of the physiological state can be determined. As additional data is acquired from the sensor array the probability of the physiological state is updated. Operating as a standalone continuous glucose monitoring system data associated with detection of a physiological state can be saved for later review in order to refine the detection algorithm. When used in conjunction with an artificial pancreas system the detection of a physiological state can be used to automatically adjust basal and bolus insulin delivery.

In many embodiments, the analyte sensor 102 is intended to be placed in subcutaneous tissue where the plurality of working electrodes within the sensor array 104a produce signals related to the analyte each transduced is configured to measure or detect (e.g., choline, glucose, tissue oxygen, lactate, ROS, ketones). In embodiments where the sensor probe 104 is intended to be placed within subcutaneous tissue, the analyte sensor 104 may also be referred to as a probe. Placement within subcutaneous tissue enables a unique perspective for an oxygen sensor that is substantially different from common SpO2 oxygen measurements. Specifically, with embodiments of the analyte sensors 104, oxygen within tissue is being measured rather than a measurement of SpO2 that is an estimation of arterial oxygen. When detecting a physiological state, it is advantageous to measure oxygen within tissue rather than estimated arterial oxygen because oxygen within tissue is a direct measurement of oxygen perfusion.

In many embodiments the analyte sensors 102 include transducers configured to measure ROS. In some embodiments, a two-electrode system is employed where each of the working electrodes electrochemically measure a particular analyte relative to a counter electrode. In other embodiments, a three-electrode system is employed where each of the working electrodes electrochemically measure a particular analyte relative to a counter and reference electrode. In one embodiment, ROS would be enabled via a pair of electrodes. An ROS measurement can be acquired through a first pair of electrodes that includes a standard working electrode and a combined counter/reference electrode, or pseudo-reference electrode. In some embodiments the ROS electrodes would provide calibration free, real-time concentration levels of oxidizing agents. In many embodiments ROS measurements may provide some insight into oxidative stress.

In some embodiments, each working electrode has a corresponding counter electrode while in other embodiments multiple working electrodes share a counter electrode. In still other embodiments, two working electrodes share a counter electrode while the third working electrode has a dedicated discrete counter electrode. Furthermore, the various embodiments of working electrodes and counter electrodes can be distributed among separate and discrete substrates. Typically, working electrodes and counter/reference electrodes are formed on a single substrate. However, an electrode design intended for use in the invention allows the complete physical separation of any of the working electrodes and any of the counter/reference electrodes. For example, as is shown in FIGS. 4A and 4B working electrodes for analyte sensors can be formed on A-side 300a while counter/reference electrodes are formed on B-side 300b. While the various electrodes may be separated on distinct A-side 300a and B-side 300b, in many embodiments the sensor array 104a having the plurality of working electrodes is inserted into the subcutaneous tissue via a single point of insertion. The use of a single insertion point minimizes both patient discomfort associated with insertion and insertion complexity.

Exemplary transducer structures that can be used for the analyte sensors 104 can be found in the following U.S. patent application Ser. No. 15/472,194 filed on Mar. 28, 2017; Ser. No. 16/054,649, filed on Aug. 3, 2018; Ser. No. 16/152,727 filed on Oct. 5, 2018; each of which are hereby incorporated by reference for all purposes. Additionally, transducer structures can be found in PCT application serial no. PCT/US18/38984 filed on Jun. 22, 2018, which is hereby incorporated by reference for all purposes.

In many embodiments, the preferred tissue to insert the probe containing the analyte sensors 104 is subcutaneous tissue. However, this should not preclude the use of the probe in other tissues such as, but not limited to skeletal muscle tissue, smooth muscle tissue or even organ tissue. Insertion of an oxygen sensor into any of these types of tissues can provide insight into the microcirculation of the specific tissue, and accordingly, the relative health of the subject.

The specific embodiments described above regarding the analyte sensors 104 and the physical sensors 110 should not be construed as limiting. In other embodiments the number of analyte sensors that can be placed on a single probe is only limited by the physical size of the probe and the willingness of subjects to insert the probe. That is to say, it should be understood that a single probe measuring choline, tissue oxygen and any number of additional analytes should be construed as being within the current disclosure. In order to accommodate additional analyte sensing electrodes it may be necessary for some analytes to share a common reference electrode. Furthermore, regarding reference electrodes, while FIG. 4B includes multiple pseudo-reference electrodes, various other embodiments can use various combinations of pseudo-reference electrodes, discrete counter electrodes, discrete reference electrodes and various combinations thereof.

FIG. 4C is an exemplary cross-section illustration of the analyte sensor 300a illustrating diffusion of analyte, reactant and reaction by-product within one working electrode in accordance with embodiments of the present invention; any sensor probe of the present disclosure may be configured to operate according to similar analyte, reactant, and/or reaction by-product diffusion as shown in FIG. 4C. The embodiment illustrated in FIG. 4C is based on the use of glucose oxidase as a first reactive chemistry 440, thereby enabling the electrode to generate hydrogen peroxide that correlates to the concentration of glucose based on the following chemical reaction:

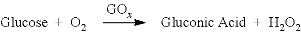

Upon insertion of the sensor assembly into a subject concentration of analytes (glucose in this illustrative embodiment) and other biomarkers around the sensor will be higher than within the individual electrodes of the sensor assembly. Concentrations of analytes and biomarkers will attempt to achieve equilibrium within the first transport materials 438 of the sensor resulting in glucose from the fluid surrounding the sensor assembly being drawn into the first transport layer 438. Fluids surrounding the sensor assembly can enter the sensor via the hydrophilic first transport material 438 but fluid cannot enter through the hydrophobic second transport material 442. In FIG. 4C glucose, represented as a ‘G’ within a hexagon, is shown entering the first transport material 438. Furthermore, oxygen, represented as ‘O₂’ is shown being supplied from the second transport materials 442 to the first reactive chemistry 440. The glucose and oxygen react with the first reactive chemistry 440 according to the chemical reaction described above resulting in the creation of by-products gluconic acid and hydrogen peroxide. The by-product hydrogen peroxide, shown as ‘H₂O₂’ in FIG. 4C, is transported via the first transport material 438 to the electrode reactive surface 416 where an applied electrical potential reduces it based on the following reaction:

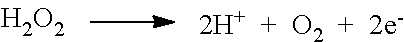

where the 2e⁻ is the electrical current picked up by the counter electrode. The consumption of glucose within the electrode lowers the concentration of glucose within the first transport layer 438 establishing a diffusion gradient that strives to reach equilibrium by bringing in additional glucose from the fluid surrounding the sensor assembly. While the above discussion is specific to glucose being detected using glucose specific enzyme, it should be readily apparent that other analytes such as, but not limited to choline, can be detected using a choline specific enzyme.

When compared to some glucose sensors that utilize a glucose limiting membrane (GLM), the electrode illustrated in FIG. 4C is easily identifiable as different in that the first reactive chemistry 440 is physically separated from the electrode reactive surface 416 by the first transport materials 438. The physical separation of the first reactive chemistry 440 from the electrode reactive surface 416 requires specific selection of the first transport material to support fundamental changes to diffusion within the electrode. For example, the first transport material 438 must support diffusion of the desired analyte in addition to the by-product of the analyte and the first reactive chemistry. Furthermore, in many embodiments it is desirable that the first transport material 438 also enables diffusion of the by-products of the electrochemical reaction occurring at the electrode reactive surface 416.

Placement of the first transport material 438 over the opening 444, and subsequent placement of the first reactive chemistry 440 over the first transport layer 438 moves the enzymatic reaction between analyte and first reactive chemistry 440 away from the electrode reactive surface 416. The separation of the enzymatic reaction and the electrochemical reaction reduces or minimizes the likelihood of localized pH fluctuations that accompany the electrochemical reaction that can have a negative impact on the first reactive chemistry 440. An additional benefit of the floating electrode is the first transport material 438 pathway that extends completely under the first reactive chemistry 440 that enables laterally diffusing analyte to be transported under and across the longest surface of the first reactive chemistry 440. After the enzymatic reaction, the by-product of the enzymatic reaction is consumed by the electrochemical reaction occurring on the electrode reactive surface 416. Accordingly, with hydrogen peroxide producing enzymatic reactions, the first transport material 438 pathway separating the electrode reactive surface 416 and the first reactive chemistry 440 enables analyte and by-products of the enzymatic reaction to move in substantially opposite directions.

An additional benefit of placing the first reactive chemistry 440 between the first 438 and second 442 transport materials is improved manufacturability. In many embodiments, the first reactive chemistry 440 is a mixture, blend or suspension of a specific enzyme, or biorecognition molecule, within a second material such as, but not limited to, the first transport material. Thus, applying the first reactive chemistry 440 over a layer of the first transport material 438 improves manufacturability because like materials are being placed on like materials.

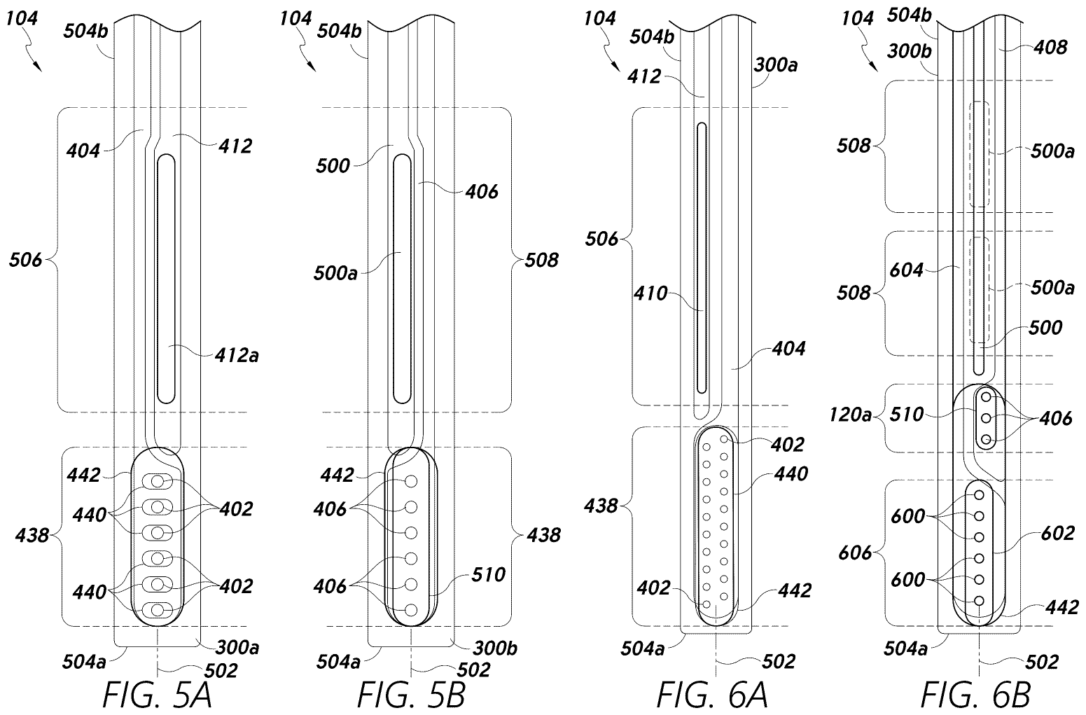

FIGS. 5A and 5B are an exemplary illustration of A-side 300a and B-side 300b of a sensor probe 104, in accordance with other embodiments of the present invention. FIG. 5A is an illustration of A-side 300a of the sensor 104 where the third electrode trace 412 supports a first counter/reference electrode. FIG. 5B is an illustration of B-side 300b of the sensor 104 where a fourth electrode trace 500 supports a second counter/reference electrode. In this embodiment, the A-side 300a includes the third electrode trace 412 that further includes third electrode opening 412a located slightly offset from the centerline 502 toward the distal end 504a. A fourth transport material 506 is applied over both the third electrode opening 412a and at least a portion of the third electrode trace 412.

On the B-side 300b is fourth electrode trace 500 that further includes fourth electrode opening 500a that is offset from the centerline 502 toward the proximal end 504b. A fifth transport material 508 is applied over both the fourth electrode opening 500a and at least a portion of the fourth electrode trace 500.

FIG. 5A, an exemplary illustration of A-side 300a of the sensor 104 where first electrode trace 404 supports first working electrodes 402. In this embodiment, the B-side 300b includes the second electrode trace 408 that further includes second electrode openings 406 located substantially on the centerline 502 of the distal end 504a. The first reactive chemistry 440 is applied discretely over each of the first electrode openings 402. Additionally, the first transport material 438 is applied over at least a portion of the first electrode trace 404 and the first electrode openings 402.

On the B-side 300b is the second electrode trace 408 that further includes the second electrode openings 406 that are formed substantially along the centerline 502 toward the distal end 504a. A second reactive chemistry 510 is applied substantially coincident over the second electrode opening 406. Additionally, a first transport material 438 is applied over at least a portion of the second electrode trace 408 and the second electrode openings. Second transport material 442 is placed over at least a portion of the first and second electrode trace 404 and 408, the first electrode openings 402, the second electrode opening 406, the first and second reactive chemistries 440 and 510 and the first transport material 438.

FIGS. 6A and 6B are an exemplary illustration of A-side 300a and B-side 300b of a sensor 104, in accordance with another embodiment of the present invention. FIG. 6A is an illustration of A-side 300a of the sensor 104 where the first electrode trace 404 supports a first working electrode and the third electrode trace 412 supports a first counter/reference electrode. In this embodiment, the A-side 300a includes the first electrode trace 404 that further includes first electrode openings 402 toward the distal end 504a and are also offset from the centerline 502. The first reactive chemistry 440 is applied contiguously over all of the first electrode openings 402 and at least a portion of the first electrode trace 404. Additionally, the first transport material 438 is applied over at least a portion of the first electrode trace 404 and the first electrode openings 402.

Co-located on the A-side 300a is the third electrode trace 412 that further includes third electrode opening 410 located offset from the centerline 502 toward the proximal end 504b. The fourth transport material 506 is applied over both the third electrode opening 410 and at least a portion of the third electrode trace 412.

FIG. 6B is an illustration of B-side 300b of the sensor 104 where second electrode trace 408 supports a second working electrode, the fourth electrode trace 500 supports a second counter/reference electrode and a fifth electrode trace 604 supports a third working electrode 410. In this embodiment, the B-side 300b includes the second electrode trace 408 that further includes second electrode openings 406 that are offset from the centerline 502. The second reactive chemistry 510 is applied contiguously over each of the second electrode openings 406 and at least a portion of the second electrode conductor 408. Additionally, the second transport material 442 is applied over at least a portion of the second electrode trace 408 and the second electrode openings 406.

Co-located on the B-side 300b is the fourth electrode trace 500 that further includes the fourth electrode opening 500a that is substantially located on the centerline 502. Note that in this embodiment, the electrode openings 500a open over a height that is greater than the height of the fourth electrode trace 500. The fifth transport material 508 is applied over both the fourth electrode openings 500a and at least a portion of the fourth electrode trace 500. Also co-located on the B-side 300b is the fifth electrode trace 604 that further includes fifth electrode openings 600 that are located substantially along centerline 502 toward the distal end 504a. A third reactive chemistry 602 is applied contiguously over each of the fifth electrode openings 600 and at least a portion of the fifth electrode trace 604.

In many embodiments, additional features or elements can be included, added or substituted for some or all of the exemplary features described above. An exemplary, non-limiting example is the use of a three electrode system (working, counter and reference electrodes) where a two electrode system (working and combined counter/reference electrodes) are discussed above. Alternatively, in other embodiments, fewer features or elements can be included or removed from the exemplary features described above. In still other embodiments, where possible, combinations of elements or features discussed or disclosed incongruously may be combined together in a single embodiment rather than discreetly or in the specific combinations described in the exemplary description found above. Accordingly, while the description above refers to particular embodiments of the invention, it will be understood that many modifications or combinations of the disclosed embodiments may be made without departing from the spirit thereof. The presently disclosed embodiments are therefore to be considered in all respects as illustrative and not restrictive.

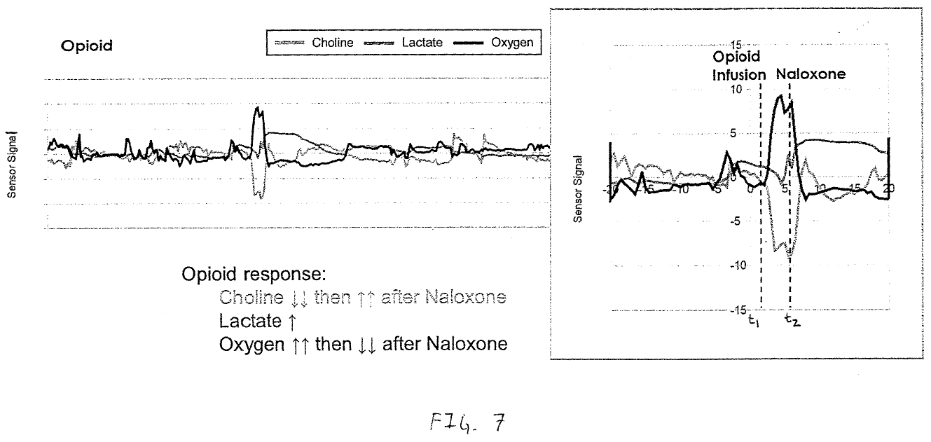

FIG. 7 is an exemplary illustration of real-time choline, lactate, and tissue oxygen data illustrative of intentional exposure to an opioid and subsequent delivery of an opioid countermeasure, in accordance with embodiments of the present invention. FIG. 7 includes illustrative choline, lactate, and tissue oxygen data with respect to time beginning at t₀. At time t₁, illustrated in the inset detail in FIG. 7, an opioid infusion is administered. This is immediately followed by a dramatic decrease in choline and a dramatic increase in signal associated with tissue oxygen. At time t₂, also illustrated in the inset detail in FIG. 7, Naloxone, an opioid countermeasure is administered. This is immediately followed by a significant increase in choline and a similar significant decrease in sign associated with tissue oxygen.

Aspects of the present disclosure provide solutions for using a sensor to measure choline and at least one other analyte such as tissue oxygen. The choline and tissue oxygen data acquired by the sensor, combined with additional data can enable the determination of exposure to opioids and/or the efficacy of opioid countermeasures. In some embodiments, the system is configured to provide warnings or notifications such as one or more of an audible, visual, and/or tactile alarm to a user or third party if threshold values for changes in both choline and tissue oxygen are detected that are indicative of exposure to an opioid.

In addition to determining if a user has been exposed to opioids, the systems of the present disclosure can further enable quantitative evaluation regarding efficacy of any opioid countermeasures that are administered to the user. In many embodiments, the multi-analyte sensor data can be used to determine if choline and tissue oxygen levels are returning to pre-opioid exposure levels as illustrated in FIG. 7 after t₂. In some embodiments, if choline and tissue oxygen levels return to pre-exposure levels, the system may automatically cancel the alarm. Additionally, in some embodiments, if the system detects exposure to opioids, a third party is notified of potential opioid exposure and the third party is also provided location information for the user. In many of these embodiments, location information may be provided by external sensors such as GPS data from a mobile device.

In some embodiments, combinations of analyte sensor data (e.g., choline and tissue oxygen) are used to initially detect a physiological state such as exposure to opioids. In other embodiments, physiological states are initially detected using a combination of analyte sensor data and physical sensor data such as, but not limited to accelerometers, temperature sensors, hydration sensors, timers, ECG and the like. In these embodiments the physical sensors are installed or enabled via hardware and/or software within the electronics package associated with the analyte sensors. In still other embodiments, initial detection of a physiological state is determined based on physical sensor data. Regardless of how initial detection of the physiological state is determined, subsequently acquired data from either analyte sensors or physical sensors can be used to confirm or reject the initial detection.

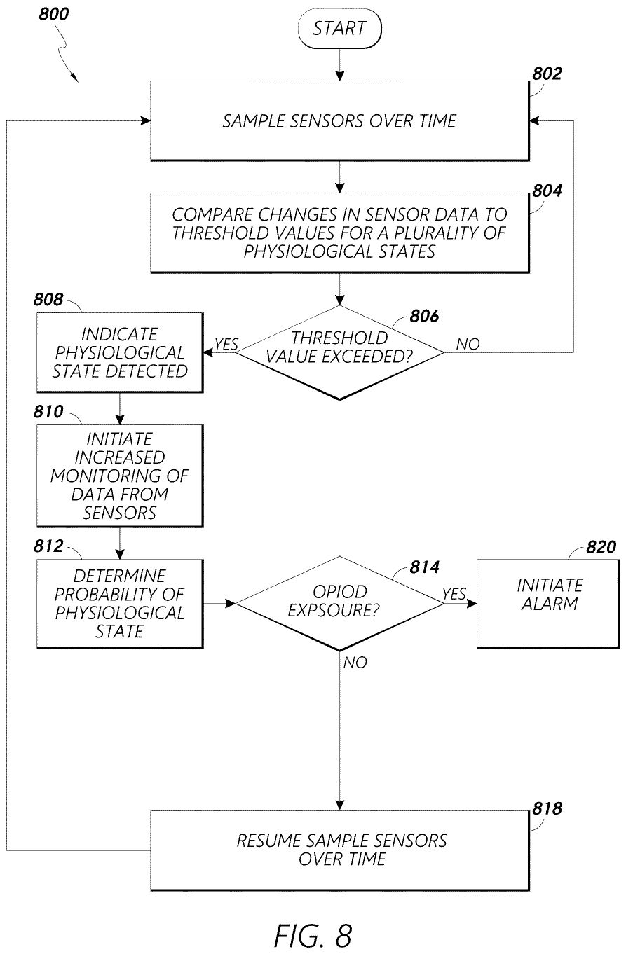

FIG. 8 is an exemplary flowchart illustrating a process 800 of detecting a physiological condition, in accordance with various embodiments of the present invention. The flowchart begins with START operation that could be viewed as being roughly equivalent to having inserted, powered, and properly hydrated, or run-in, the analyte sensor. Additionally, in some embodiments, physical sensors may be similarly ready to produce meaningful data. Operation 802 can involve sampling and storing data from both the analyte sensors and physical sensors. Operation 804 may further involve comparing changes in data streams from the sensors to threshold values associated with exposure to opioids. For example, at operation 804, data streams from the analyte and physical sensors may be processed to determine combinations of absolute changes or rates of change for choline and tissue oxygen that exceed a threshold value indicative of opioid exposure.

Operation 806 involves comparing whether predetermined threshold value(s) indicative of opioid exposure have been exceeded. If no threshold values have been exceeded, the process 800 may return to operation 802. If a threshold value has been exceeded, the process 800 may proceed to operation 808 which indicates a physiological state may be detected. For example, in some instances a choline threshold (e.g., absolute value and/or rate of change) may be exceeded while the tissue oxygen threshold has not been exceeded. Because of the potential physiological state of opioid exposure associated with the choline threshold being exceeded the flowchart proceeds to operation 810. Operation 810 may involve initiating increased monitoring of data from the sensors. With a physiological state detected, the system can begin interrogating analyte and physical sensors to obtain data to either confirm or refute the initial detection. For example, the sampling rate for one or more of choline and/or tissue oxygen may be increased from once every 10 seconds to once every two seconds.

Operation 812 can involve determining or calculating a probability of the physiological state based on the increased monitoring from the sensors. For example, operation 812 can involve evaluating tissue oxygen and choline data to determine if both choline and tissue oxygen levels (either absolute values and/or rate of change) have exceeded their threshold values. Operation 814 evaluates if both the choline and tissue oxygen data are indicative of opioid exposure. Operation 820 initiates an alarm if operation 814 determines there has been exposure to opioids. While operation 818 resumes sampling of sensor over time if operation 814 determines there as not been exposure to opioids.

Throughout the description the system has been described as sensors associated with an electronics package that enable communication to additional devices. However, in some embodiments, the system 100 (FIG. 1) can be incorporated into a network of sensors that includes various combinations of multiple sensors (ranging from non-invasive, minimally invasive to invasive) that are optionally interconnected via an on-body network. The networked sensors can be similar to those described in U.S. patent application Ser. No. 15/417,055 filed on Jan. 26, 2017 and Ser. No. 16/054,649, filed on Aug. 3, 2018. Alternative embodiments utilize a variety of other sensors capable of measuring a variety of other conditions. Each different sensor within the networked sensors can be operated continuously or alternatively, periodically or episodically to gain additional insight into physiological states and conditions.

Accordingly, while the description above refers to particular embodiments of the invention, it will be understood that many modifications may be made without departing from the spirit thereof. In particular, while many embodiments are directed toward specific combinations of analytes and physical sensor data, it should be understood that where possible, each embodiment is capable of being combined with each and every other embodiment. The presently disclosed embodiments are therefore to be considered in all respects as illustrative and not restrictive, the scope of the invention being indicated by the appended claims, rather than the foregoing description, and all changes that come within the meaning and range of equivalency of the claims are therefore intended to be embraced therein.

Although certain preferred examples are disclosed above, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, various examples may be carried out in a manner that achieves or optimizes one advantage or group of advantages as taught herein without necessarily achieving other aspects or advantages as may also be taught or suggested herein.

Certain spatially relative terms, such as “outer,” “inner,” “upper,” “lower,” “below,” “above,” “vertical,” “horizontal,” “top,” “bottom,” “distal,” “proximal,” and similar terms, are used herein to describe a spatial relationship of one device/element or anatomical structure to another device/element or anatomical structure. It should be understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

Certain reference numbers are re-used across different figures of the figure set of the present disclosure as a matter of convenience for devices, components, systems, features, and/or modules having features that are similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

What is claimed is:

1. A multi-analyte sensor system comprising:
a sensor probe comprising:
a first set of electrodes including one or more first working electrodes configured to transduce choline into electrical signals;
a second set of electrodes including one or more second working electrodes configured to transduce tissue oxygen into electrical signals; and
a third set of electrodes including one or more third electrodes that provide working and counter electrode functionality for the first set of electrodes and the second set of electrodes;
an electronics module configured to electrically interface with the sensor probe, the electronics module including a transceiver configured to transmit sensor data; and
control circuitry communicatively coupled to the electronics module and configured to:
determine a choline state based on one or more signals from the first set of electrodes;
determine a tissue oxygen state based on one or more signals from the second set of electrodes; and
initiate an alarm condition based on the one or more signals from the first set of electrodes and the one or more signals from the second set of electrodes.

2. The multi-analyte sensor system of claim 1, wherein the control circuitry is a component of the electronics module.

3. The multi-analyte sensor system of claim 1, wherein the alarm condition is generated on a local device.

4. The multi-analyte sensor system of claim 3, wherein the alarm condition is generated on a remove device.

5. The multi-analyte sensor system of claim 1, wherein:
the sensor probe further comprises a fourth set of electrodes including one or more third working electrodes configured to transduce glucose into electrical signals.

6. The multi-analyte sensor system of claim 1, wherein the sensor probe is configured to detect tissue impedance.

7. The multi-analyte sensor system of claim 1, further comprising an accelerometer associated with the electronics module.

8. A continuous multianalyte monitoring system, comprising:
a skin-mounted sensor control unit, comprising:
a percutaneous multianalyte sensor including an insertion portion configured for transcutaneous positioning in a subcutaneous tissue of a user, the percutaneous multianalyte sensor being configured to sense levels of choline and tissue oxygen in the subcutaneous tissue of the user; and
an adhesive patch disposed on a bottom surface of the skin-mounted sensor control unit and configured to adhere the skin-mounted sensor control unit to skin of the user;
a transceiver configured for wireless communication with the skin-mounted sensor control unit; and
control circuitry configured to:
receive and store signals from the percutaneous multianalyte sensor related to sensed levels of choline and tissue oxygen;
determine a real-time physical condition value based on the received signals related to sensed levels of choline and oxygen;
determine whether the user has been exposed to an opioid based on the real-time physical condition value; and
generate user interface data for rendering on a touch-interface display to visually display a graph of the choline and tissue oxygen levels, the graph representing a first axis corresponding to time, a second axis corresponding to one or more of the choline levels or tissue oxygen levels, and the physical condition value.

9. The continuous multianalyte monitoring system of claim 8, wherein the control circuitry is further configured to maintain historical real-time insulin condition values in data storage of the control circuitry.

10. A multi-analyte sensor system comprising:
a sensor probe comprising:
a first set of working electrodes configured to transduce choline into first electrical signals;
a second set of working electrodes configured to transduce tissue oxygen into second electrical signals; and
a third set of electrodes that are configured to provide reference and counter electrode functionality for the first set of working electrodes and the second set of working electrodes;
an electronics module configured to electrically interface with the sensor probe, the electronics module including a transceiver configured to wirelessly transmit sensor data; and
control circuitry communicatively coupled to the electronics module and configured to:
store threshold values that are indicative of opioid exposure;
determine a choline state based on one or more signals from the first set of working electrodes;
determine a tissue oxygen state based on one or more signals from the second set of working electrodes;
determine an opioid condition based on at least the choline state and the tissue oxygen state, and
initiate an alarm condition based on the opioid condition.
